(19) United States
(12) Patent Application Publication
(10) Pub. No.: US 2002/0028156 A1
(43) Pub. Date: Mar. 7, 2002

(54) Composite oxygenator membrane

(76) Inventors: Bruce L. Anneaux, Clemson, SC (US); Shalaby W. Shalaby, Anderson, SC (US); R. Larry Dooley, Clemson, SC (US)

Correspondence Address: LEIGH P. GREGORY, ATTORNEY AT LAW, PO BOX 168, CLEMSON, SC, 29633-0168, US

(21) Appl. No.: 09/907,136

(22) Filed: Jul. 17, 2001

Related U.S. Application Data
(60) Provisional application No. 60/218,917, filed on Jul. 18, 2000.

(51) Int. Cl. A61M 1/14

(52) U.S. Cl. 422/48

(57) ABSTRACT

This invention deals with a composite membrane comprising a thermoplastic matrix and fibrous reinforcing construct for use in constructing membrane blood oxygenators. The surface of the composite membrane can be chemically activated to incorporate functional groups to provide certain desirable properties to increase the utility of the membrane and extend its use to chromatographic applications and incorporation in dialysis units.

### BACKGROUND TO THE INVENTION

The current designs of blood oxygenators provide acceptable performance and blood handling characteristics to be used for a wide variety of surgical operations requiring cardiopulmonary bypass (CPB). They do, however, provide these functions at a substantial cost to the immune, complement activation and coagulation systems of the human body. With a surface area of 2-4.6 square meters, the oxygenator presents a direct contacting foreign surface area larger than any other medical device on the market. Their prime volume results in a hemodilution of 8 to 20% for the average adult patient undergoing CPB. The oxygenators on the U.S. market to date are primarily for use in surgeries requiring less than five hours of CPB employing moderate to deep hypothermia. The membrane design of these oxygenators is a microporous one that succumbs to the constant pressure and deposition of plasma proteins and finally begins to leak plasma water from the blood phase to the gas phase of the unit, which greatly compromises efficiency and reliability. Current surgical techniques in terms of depth of hypothermia, speed and decreases depth of anesthesia will begin to push the envelope of efficiency for currently used oxygenators as decreased depth of hypothermia and anesthesia together raise the metabolic demand of a typical patient requiring CPB.

The control of oxygen transfer across artificial membranes for means of providing pulmonary support has been one of the primary areas of focus in oxygenator design since the first one was made. The challenge is in making an efficient, biocompatible and strong membrane all at once. The efficiency is governed by the material used, the thickness and the porosity of the membrane. An adult blood oxygenator must be capable of raising a venous blood oxygen saturation of 65±5% to greater than 97% at a rated flow of 6 liters per minute, a hemoglobin concentration of 12±1 g/dl at 37±2° C. During most CPB procedures the blood pressure seen at the oxygenator membrane can reach as high as 450 mmHg. With this in mind, the membrane must be as strong as possible and still maintain performance. However, the efficiency of the membrane is inversely proportional to the thickness while the strength, in most cases, is directly proportional to the thickness. Consequently, several new materials have been explored for use as oxygenator membranes. Most new materials are designed to provide true membranes as opposed to the more commonly used micro-porous polypropylene membranes which exhibit plasma leakage with long-term usage

Examples of the polymeric systems used as membrane components to modulate gas transfer include fluorinated polyimides and poly-4-methylpentene membranes and silicone coated hollow fibers.

Fluorinated polyimide membranes provide acceptable gas permeability and biocompatibility. The method used to prepare the membranes results in the formation of a very thin (20 nm) skin layer that eliminates the occurrence of plasma leakage. The underlying support for the skin layer is an open cell type matrix which offers little resistance to gas diffusion. The oxygen and carbon dioxide transfer rates are very good and the membranes induce little to no platelet aggregation when tested in vitro. However, these films are sub-optimal in terms of compliance and elasticity.

Poly-4-methylpentene (PMP) is used as a hollow fiber membrane which produces an asymmetric geometry like the fluorinated polyimide mentioned earlier. The structure consists again of a foam-like core and an outer skin with a thickness of less than one micron. The asymmetric structure of this fiber is achieved using controlled melt spinning and drawing conditions. The thin skin allows for good gas transfer while eliminating the plasma leakage common with microporous membranes when used long term. However, the PMP hollow fibers are highly sensitive to oxidation which can result in rapid deterioration in their mechanical properties. Additionally, the PMP hollow fibers are sub-optimal in terms of elasticity and compliance.

Expanding on the overall wide acceptance and adequate performance for short-term usage of the micro-porous hollow fiber membranes, a recent system was developed that consists of a 0.2 micron coating of silicone over a standard micro-porous hollow fiber. The characteristics of the silicone membrane were found to be advantageous because of the relatively high oxygen transfer capabilities and the absence of plasma leakage associated with this membrane construct. The final product of this hybridization is a hollow fiber oxygenator with no plasma leakage. However, in addition to the questionable mechanical stability of silicone coating and its interface with the hollow fiber substrate, the coating lowers the gas transfer efficiency of the hollow fiber membranes if conventional hollow fibers are used.

The oxygenators in clinical use today offer a much improved solution to CPB over the spinning disc oxygenator that existed fifty years ago. Current oxygenators are relatively more efficient and biocompatible. There are still, however, major improvements that could be made to the design and composition of the oxygenator membranes. To address this, it must be recognized that the primary deficiencies that exist with all micro-porous oxygenators include plasma leakage if used over five hours, large surface areas of foreign material, prime volumes from 2.2-4.2 m², and a high level of blood component activation and consumption. Although the majority of the time periods on CPB are less than three hours, it is desirable to have an oxygenator that can provide the efficiency needed for short term operations and still eliminates the possibility of plasma leakage.

Toward the development of a novel oxygenator membrane, the following performance parameters were taken into consideration:

Plasma Leakage

The outside evidence of plasma leakage is a frothing exiting the gas exhaust port on the oxygenator. Even before this is present, there is a reduction in the efficiency of the membrane as proteins continue to coat the membrane surface. The initial stages, i.e., the presence of water vapor on the gas side of the membrane, are not visible to the operator and therefore go unnoticed as a precursor of what is sure to follow. With no arterial oxygen saturation monitor, the gradual drop in oxygenator efficiency may give the appearance that the patient is not anaesthetized deeply enough or the patient is hyper-metabolic and may need to cooled down to a cooler temperature to adequately lower the metabolic rate. It is this uncertainty and masked drop in oxygenator performance that causes plasma leakage to become such an important target for improvement in oxygenator performance.

Sub-optimal Permeability Profile

Traditional oxygenator membranes provide adequate gas transfer for routine surgeries involving CPB. However, their performance is inversely proportional to the duration of use. The least desirable feature of microporous membranes is associated with their microporosity, as the presence of these pores leads to compromised performance and increased blood component activation. As surgeries become shorter with better surgeon training and more efficient operating techniques, the degree of hypothermia required for CPB will decrease as will the depth of anesthesia. Managed medicine is pushing for shorter hospital stays and faster recovery periods for even major surgery such as coronary artery bypass grafting (CABG) and valve repair/replacement. Faster recovery time translates to faster extubation and ambulation that ultimately requires “fast track anesthesia.” This method of using smaller doses of traditional narcotic analgesics and/or shorter acting anesthetics presents a patient whose metabolic demand is greater and, therefore, requires greater gas transfer to sustain normal tissue functions. The oxygenators in use today will begin to reach the edge of their envelope of efficiency as less hypothermia and less anesthesia together raise the metabolic demand of typical patients requiring CPB. The maximum oxygen transfer required by ISO and Association for the Advancement of Medical Instrumentation (AAMI) guidelines is currently 4.5 mlO₂/100 ml blood. For an average adult at rest, the oxygen consumption is 140 mlO₂/m² and the cardiac output is 5 L/min. Using an average body surface area for adults of 1.7 m², the O₂ demand is 238 mlO₂/min and the AAMI/ISO guideline provide for 225 mlO₂/min. Although one would expect to see lower O₂ consumption with an anesthetized adult, the stress associated with surgery can raise the body's oxygen requirements. Based on these calculations and anecdotal evidence, the efficiency of today's oxygenators will begin to be challenged.

Blood Constituent Activation

The current designs of blood oxygenators provide adequate performance allowing their use in a wide variety of surgical operations requiring CPB. They do, however, provide these functions at a substantial cost to the immune, complement activation and coagulation systems of the human body. Collectively, the activation of these systems is accountable for most of the complications associated with CPB such as induced consumptive coagulopathies, neurological changes and immune system disorders. While most of these problems are reversible after CPB, they still pose interim problems that require medical intervention in some cases.

High Foreign Surface Area to Prime Volume Ratio

With a surface area of 2-4.6 m², the oxygenator is the largest blood contacting medical device on the market. It is this large foreign surface area that is responsible for the depth of problems associated with CPB. A reduction in surface area will provide an equal reduction in the disruption to the biological systems that are targeted by CPB. Until a purely biological solution can be developed to meet the challenge of designing a truly biocompatible oxygenate, efforts must be made to improve on the biocompatibility of currently available materials and to develop new ones.

Recognizing the aforementioned drawbacks associated with commercially available membrane oxygenators and candidate membranes reported in the prior art provided an incentive to develop a novel system with superior gas transfer characteristics, reduced surface area and improved hem compatibility. This invention deals with novel composite membranes for use in constructing biocompatible, high efficiency membrane oxygenators.

### SUMMARY OF THE INVENTION

The present invention is directed to a composite thermoplastic membrane for use primarily in membrane blood oxygenates which includes a polymeric matrix and a reinforcing fiber-based material. The polymeric matrix of the composite is formed from a thermoplastic Eastover such as polyolefin-polydiene copolymers, hydrogenated polyolefin-polydiene copolymers, polystyrene, polydiene copolymers, polyether-urethane copolymers, polyether-ester copolymers, polydimethyl siloxane-polyether urethane copolymers, polyether-polyolefin copolymers, or blends thereof. The reinforcing fibers of the composite can be in the form of woven, non-woven, or knitted fabrics. The matrix of the composite can be plasticized to maximize its permeability. The polymeric matrix of the composite membrane can be crosslinked following its assembly to improve its mechanical properties. The surface of the composite membrane can be chemically activated to covalently bind phosphonyl or sulfonyl moieties that can be used to broaden the scope of the membrane applications to include its use in chromatography, dialysis and blood purification as well as separation and purification of proteins and fractionation of bioactive agents, such as drugs, peptides, and proteins.

### BRIEF DESCRIPTION OF THE FIGURES OF THE DRAWING

FIG. 1 is a graph illustrating an oxygen transmission rate curve for two similar films made in accordance with the present invention but dried by different methods;

FIG. 2 is a graph illustrating an oxygen transmission rate curve for a film made in accordance with the present invention;

FIG. 3 is a graph illustrating an oxygen transmission rate curve for a film made in accordance with the present invention;

FIG. 4 is a graph illustrating an oxygen transmission rate curve for a film made in accordance with the present invention;

FIG. 5 is a graph illustrating an oxygen transmission rate curve for a film made in accordance with the present invention;

FIG. 6 is a graph illustrating an oxygen transmission rate curve for a comparative membrane;

FIG. 7 is a graph illustrating an oxygen transmission rate curve for a comparative membrane;

FIG. 8 is a graph illustrating an oxygen transmission rate curve for a film made in accordance with the present invention;

FIG. 9 is a graph illustrating an oxygen transmission rate curve for a film made in accordance with the present invention;

FIG. 10 is a graph illustrating an oxygen transmission rate curve for a film made in accordance with the present invention;

FIG. 11 is a graph illustrating an oxygen transmission rate curve for a film made in accordance with the present invention;

FIG. 12 is a graph illustrating an oxygen transmission rate curve for a film made in accordance with the present invention;

FIG. 13 is a graph illustrating an oxygen transmission rate curve for a film made in accordance with the present invention;

FIG. 14 is a graph illustrating an oxygen transmission rate curve for two similar films made in accordance with the present invention but dried by different methods;

FIG. 15 is a graph illustrating an oxygen transmission rate curve for a film made in accordance with the present invention;

FIG. 16 is a graph illustrating an oxygen transmission rate curve for a film made in accordance with the present invention;

FIG. 17 is a graph illustrating oxygen transmission rate curves for two films made in accordance with the present invention and a comparative membrane.

### DETAILED DESCRIPTION OF PREFERRED EMBODIMENTS

This invention deals with a composite membrane for use in constructing an oxygenator, wherein the membrane is a polymeric matrix of a thermoplastic elastomer (TPE) reinforced with a fibrous construct. The fibrous reinforcement comprises a woven, non-woven, knitted or braided material made typically of a polyester or polyolefin. The thermoplastic elastomer can be a diene/olefin block copolymer such as poly(butadiene-b-polystyrene), poly(isoprene-b-styrene) and/or their hydrogenated forms. The TPE can also be a member of the segmented polyether-esters, polyether urethanes or polyester urethanes. These polymers meet the basic requirements of a compliant, elastic film former that is (1) amorphous or has relatively low crystallinity as compared to traditional crystalline thermoplastics such as PMP, (2) more flexible and elastic than polyimides and PMP, (3) higher in tear strength than silicone, (4) not known to have hemocompatibility or toxicity problems, and (5) easily processed.

In effect, the commercial materials selected for use in film casting to form the polymeric matrix of the present inventive composite membrane were polyether urethanes sold under the tradename Tecoflex® by Thermedics Polymer Products (Woburn, Mass.), segmented copolymers of poly(oxytetramethylene glycol) and poly(tetramethylene terephthalate) sold under the tradename Hytrel® by DuPont Engineering Polymers (Wilmington, Del.), and styrene-butadiene-styrene block copolymers sold under the tradename Kraton® by the Shell Chemical Co. (Houston, Tex.). Tecoflex® is a historically significant medical grade polymer with many applications. It is a thermoplastic elastomer (TPE) with good strength and elasticity. It is soluble in a wide range of solvents and is available in special grades for solvent casting. Studies have shown Tecoflex® to have good hemocompatibility and no toxicity. It does, however, contain polyether blocks, which are moderately interactive with oxygen. Hytrel® has medical usage in a commercially available suture and is also a TPE with good strength and elasticity. Hytrel® can be dissolved in both chloroform and methylene chloride at concentrations which produce good results when solvent casting. It is not available in solution grades but can be found in powdered form which facilitates dissolution. Hytrel® also contains polyether blocks. Kraton® is a high molecular weight amorphous polymer with high tear strength. Kraton® is also a very stable carbon-based homochain polymer available in hydrogenated isoprene-stryrene or butadiene-styrene block copolymeric structures. It can be plasticized with polyolefinic oils which add a degree of versatility in gas transfer control. Kraton® was found to be soluble in both chloroform and dichloromethane (DCM).

Alternatively, polydimethylsiloxane segment or block copolymers may be employed as the thermoplastic elastomer of the present polymeric matrix. Regardless of the TPE employed, the polymeric matrix of the present composite optionally may be crosslinked following composite assembly. Further, the TPE may include a plasticizer to increase membrane permeability, as is discussed in greater detail below.

In accordance with the present invention, the polymeric matrix includes a fibrous reinforcement to provide additional mechanical strength without compromising to a significant extent the flexibility, elasticity and permeability of the basic membrane. The fibrous construct can be woven, non-woven, knitted or braided (including melt blown materials). It is also within the scope of the present invention to use fibrous constructs that are compatible with the membrane material and deform in concert with incremental deformation of the basic membrane during actual use.

It is also within the scope of the present invention to render the surface of the basic membrane negatively charged to minimize or eliminate platelet adhesion. Such a surface modification is achieved by subjecting the composite construct to gas phase phosphonylation followed by hydrolysis. It is further within the scope of this invention to control the phosphonylation process to allow the development of microporous and/or nanoporous structures in the basic membrane material. Further details of this invention are illustrated by the examples given below.

In addition to their use as blood oxygenators, the present inventive composites may be employed as part of a chromatographic unit for the separation or purification of bioactive agents including peptides and proteins. Further applications of the composite membrane include being part of blood purification or dialysis units.

### EXAMPLES

The following techniques were employed in preparing and testing composite oxygenator membranes in accordance with the present invention:

Film Formation

Polymer solutions were produced on a flat solvent casting bed using a rod having grooves at each end, that rides on tracks on either side of the bed. The depth of the grooves in the rod were adjusted to vary the thickness of the cast solution and, thereby, the thickness of the polymer membrane following evaporation of the solvent. The variation in depth of the groove in the drawing rod was “mapped” using a 12.5% w/v solution of 4056 Hytrel in chloroform. The end of the rod was etched to indicate a reference point and multiple films were cast using varied rotations of the rod. Once dried thoroughly, representative samples were measured for thickness and referenced to the corresponding position of the reference mark. Prior to casting films, the casting plate and rod were cleaned thoroughly with acetone and dried with Kimwipes to ensure a clean and dust free surface. Films were subsequently made by securing a piece of release paper to the casting plate using lab tape and pouring a line of polymer solution (approximately 2 cm by 16 cm) across one narrow end of the release paper. After wrapping the drawing rod for protection, the notches of were sprayed lightly with silicone mold release to ensure smooth motion. The drawing rod was then pulled across the solution to the opposite end of the casting plate using a uniform pressure and constant speed.

Film Drying

Once films were cast, they were dried using two different methods. The first was complete air drying at room temperature for not less than fourteen hours. The second method was to allow the film to dry in room air for five minutes to allow for evaporation of a large percentage of the solvent and then to apply low heat with an infrared lamp placed twenty inches above the film and release paper for thirty minutes. The film was then allowed to remain at room temperature for not less than eight hours. All drying was performed in a certified fume hood.

Thickness Measurements

Once films were completely dried, representative samples were taken from four locations on the film outside the area to be tested. The samples were attained by using a 15/16″ punch. After the samples were punched, the film was removed and weighed for all four samples. The average of the weight was taken and used along with the specific gravity and area of the punched samples to calculate the average sample thickness. Any samples with variations of more than 2.5μ between the four samples were discarded.

Oxygen Permeability Measurement

The oxygen transmission rate was determined using a process that was modified from ASTM D3985-95 and ASTM D1434-82. The films to be tested for oxygen transmission rates were affixed to a poster board support frame using double-sided tape to prevent them from drawing together when removed from the release paper. The square frame measured 6.5 inches on the outside edge and 4.5 inches on the inside edge. The films were affixed to the frame without removing them from the release paper. The film to be tested was mounted on the bottom half, or monitored half, of the test cell. The top half, or source half, was then placed over the film and the two were bolted together sealing the film in place with the “O” rings embedded in each half. Once sealed, the needle valves on both halves were opened and the unit was purged with nitrogen until the oxygen concentration was below 0.2%. Once this was attained, the nitrogen purge was discontinued and the needle valve on the bottom half was closed. The purge through the top half was then switched to oxygen and the testing was started. Samples were taken through a septum in the bottom half at ten minute intervals and injected immediately into the OXTRAN unit for analysis. The concentration of oxygen was recorded in %O₂. The sampling was repeated until the %O₂ reached 90% or the volume of oxygen transmitted across the membrane per unit time was less than 2%/10 minutes. The resulting gas transmission rate was calculated by knowing the volume of the bottom half of the test cell and the %O₂ change over time.

Composite Membrane Assembling

After testing the gas transmission rate (GTR) of all the films, several were selected to be used in the composition of the composite membrane construct. The criteria for selection was a GTR that was comparable to or greater than that of the comparative silicone membrane, the 0800 ECMO Extended Capacity Membrane Oxygenator sold by Avecor Cardiovascular (Minneapolis, Minn.). These films were combined with a thin open knit polyester fabric by “gluing” the film to the fabric using a dilute solution of the polymers used in the film manufacturing. A 10:1 dilution of the polymer solution used in casting the films was made. A light coat of the dilute solution was sprayed onto the fabric and allowed to dry for 60 seconds. The film then placed over the fabric and pressed with a one-kilogram weight for 30 minutes. The composite membranes were removed and tested in the same manner as the films.

An alternative approach for forming the membrane involved hot pressing the films onto the fabric with pressure and heat, although this proved to be a less desirable method under the conditions employed. Similarly, a promising method for forming membranes in accordance with the present invention, which was less preferred under the conditions employed, was the process of actually casting the polymeric matrix directly onto the reinforcing fiber-based material.

Gas Phase Phosphonylation

Phosphonylation was conducted following a method described in U.S. Pat. No. 5,491,198. In this process, the item to be phosphonylated is suspended above a reservoir containing phosphorous trichloride in an oxygen rich environment. The films used in this study were much thinner than the typical films or sheets which receive this treatment. Because of this, much smaller quantities of PCl₃ (<0.5 ml) were used and the processing time was reduced greatly to approximately one minute. Once films were treated, they were removed to a container with constant flowing tepid water to facilitate hydrolysis. This process results in the formation of polyolefin-phosphonic chlorides on the surface of the polymer being treated.

Pressure Testing

To perform the pressure test, the films along with the support matrix were mounted on one half of the test cell and an open ring was placed on top of the film to seal the edges. The lower half of the test cell was then fitted with a 400 mmHg pressure gauge and an inlet line connected to a 60 cc syringe. The cell was filled with water and all air evacuated. The bottom cell was then pressurized to 400 mmHg and held constant for fifteen minutes. If after fifteen minutes, there was no sign of leaking, the membrane and it's corresponding permeability data were considered acceptable. In the event of leakage, the membrane and its test data were discarded and a new one made and tested.

Composition of Candidate Polymer Solution for Casting

First, the subject polymer was tested for solubility and the maximum attainable concentration in each solvent was determined. This was to maximize the casting efficacy and film uniformity through minimal dependence on solvent evaporation rate. The solvents used were chloroform and dichloromethane. Once a solution of appropriate viscosity was achieved, several test castings were made to determine if the concentration was appropriate. The ideal concentration and viscosity were found to be one at which the solution could be easily poured but viscous enough to have minimal spreading when poured on the release paper. The cast solution was observed for uniformity in spread, time for drying and absence of deformities (“fish eyes” and streaks). The type of solvent and the concentration of polymer were found to greatly influence the drying time of the film which affected the oxygen transmission rates at testing. The type of solvent used to form the polymer solution affected the resulting film quality by the speed at which it evaporated. Although higher concentrations of polymer could be attained using DCM, the evaporation rate made it difficulty to use in solvent casting and obtain a smooth defect free film. Even though very smooth surfaces were present on both the release paper and drawing rod, there were still minor irregularities on the film surface immediately after casting. When DCM was used as the solvent, it evaporated at such a rate as to prevent the solution from settling and forming a smooth defect free surface. It was therefore beneficial to use a solvent with a slower rate of evaporation, namely chloroform. The one exception to this was the harder 4056 Hytrel®. The solubility of this grade Hytrel® in chloroform was not high enough to produce a solution that yielded a film of good quality and acceptable thickness. It was therefore dissolved in DCM. The concentrations found to produce the best results for each polymer are listed in Table I. Example numbers are provided for the resultant films and, where appropriate, their eventual composite membranes. Comparative Example 7 is the commercially available silicone membrane, which was employed as the permeability standard, as described above.

TABLE I
Polymer Solution Concentrations

| Film of | Polymer | Solvent | Concentration (w/v) |
|---|---|---|---|
| Example 1 | 3078 Hytrel ® | Chloroform | 12.5% |
| Example 2 | 4056 Hytrel ® | DCM | 12.5% |
| Example 3 | 80A Tecoflex ® | Chloroform | 15% |
| Example 4 | 93A Tecoflex ® | Chloroform | 14% |
| Example 5 | 2104 Kraton ® | Chloroform | 22% |
| Example 6 | 1101 Kraton ® | Chloroform | 12.5% |
| Comparative | Avecor 0800 ECMO | | |
| Example 7 | | | |

Film Preparation and Effects of Processing on Permeability

The process of creating thin films by solvent casting proved to be a very labor intensive and exacting process. To create films with a thickness if 10-25μ, all aspects of the casting process were required to be monitored closely. The fume hood in which the casting was performed required thorough cleaning to eliminate the chance of dust or debris settling on the films as they dried. Films that were discarded due to pinhole leaks were viewed under a microscope and found to contain some form of contamination at the site of the hole. In addition to the aforementioned method of controlling film thickness by rotation of the non-concentric drawing rod, it was noticed that the speed at which the drawing rods were pulled across the solution affected the thickness of the film. The film thickness was found to be directly proportional to the speed at which the polymer solution was spread. The rationale behind this being that the faster the drawing rod was moved, the greater the force and shear between the rod and solution. This interaction served to force the solution under the drawing rod thus increasing the thickness of the polymer solution which was cast on the release paper. To eliminate this problem, the time to draw the rod from one end of the plate to the other during the casting process was regulated as closely as possible to eight seconds. In addition, the friction between the aluminum draw rod and the rails on the side of the plate caused binding and uneven drawing of the solution. This resulted in rippled films and non-uniform thickness. To eliminate this problem, silicone mold release spray was used to apply a light coat of lubricant to the notches of the drawing rod. This provided smooth and even drawing and eliminated the ripples and differences in thickness. After the films were cast and dried, they were fitted with a support. The support frame prevented the thin films from collapsing on themselves when they were removed from the release paper to be tested. The frame was attached to the surface of the film while it was still on the release paper using double-sided tape. This also provided a means of handling the films without touching the surface and affecting the GTR with skin oils and dirt. Following GTR testing, the films, along with the attached frame, were removed from the test cell and placed back on the same piece of release paper.

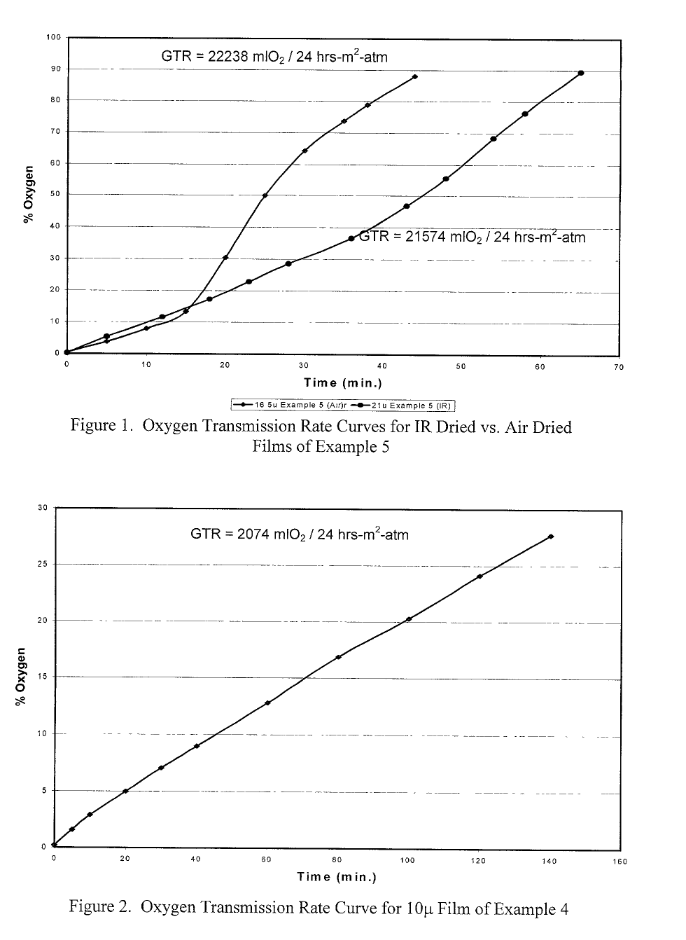

Another processing variable that had a notable effect on the films was the method in which they were dried. In the later stages of research, an infrared (IR) lamp was used to speed the drying process of the Kraton® films (the films of Examples 5 and 6). It was observed that the films dried using the IR lamp possessed different gas transmission rate (GTR) curves. The GTR curves of the films dried using the IR lamp had a gradually increasing slope whereas the films dried under ambient lab conditions experienced a marked increase in GTR after the first twenty minutes. As can be recognized by the GTR rate curves for the two drying schemes shown in FIG. 1, the final GTR was very similar. This difference in the slopes of the curves is attributable to the formation of a high-density skin on the film surface when dried using the IR heat source. The properties of the skin varied from that of the bulk material and therefore caused a difference in the initial oxygen diffusion across the films.

Oxygen Permeability Testing and Gas Transmission Rates of Unreinforced Membranes

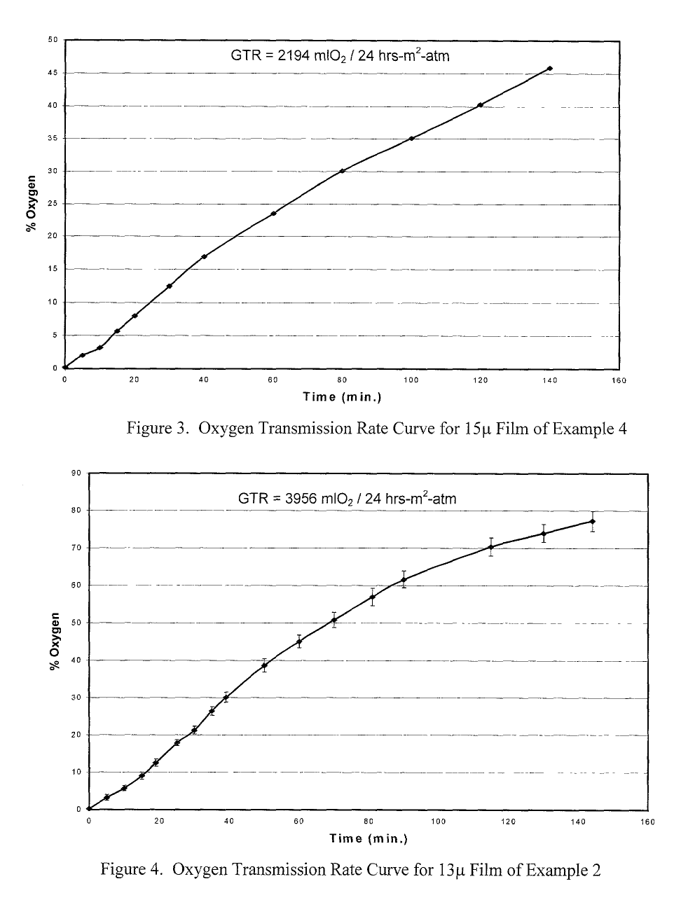

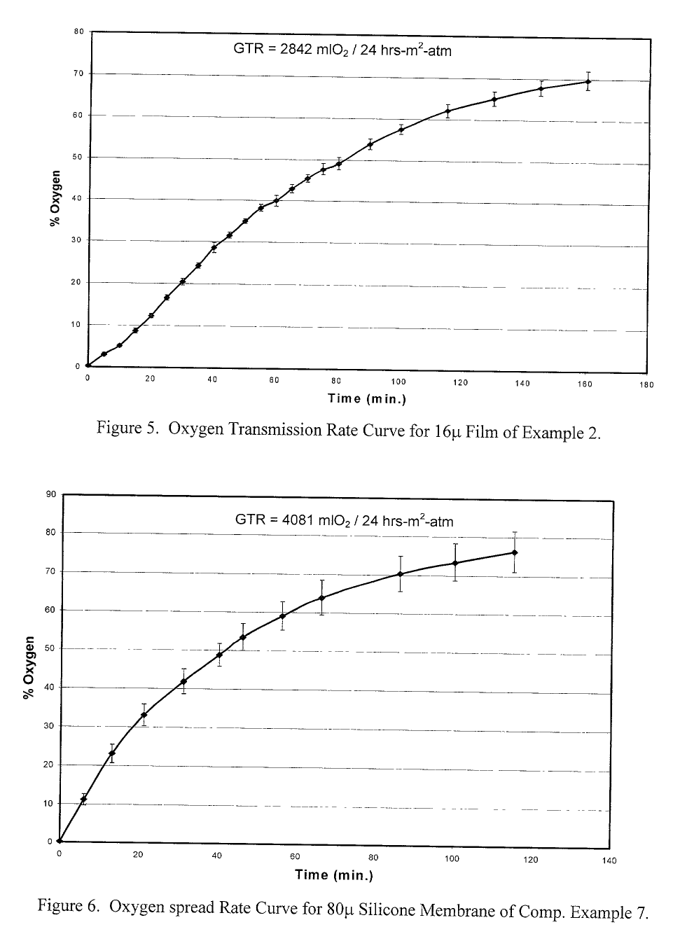

The procedure for testing the oxygen permeability of the films was adapted from ASTM standards D3985-95 and D1434-82 and procedures discussed in several packaging science publications. The procedure discussed hereinabove was relatively simple to execute and required little equipment. The Oxtran unit used displayed percent oxygen content of the injected gas via a digital readout. Although there were several modern GTR testers available, none were capable of providing results for the high level of oxygen permeabilities inherent to the films made for this study. As is the case with any gas transmission testing apparatus, the most critical and difficult task is ensuring the absence of any gas leaks. To do this, all gas line fittings, sampling septa and valves were checked for leaks using a mild detergent solution. The solution was applied to all connections and observed for the formation of bubbles which would indicate a leak. Following the elimination of the gas line leaks, the gaskets which sealed the film in placed were inspected for pliability and defects and replaced as needed. FIGS. 2-5 illustrate the oxygen transmission rate curves for the films of Examples 2 and 4.

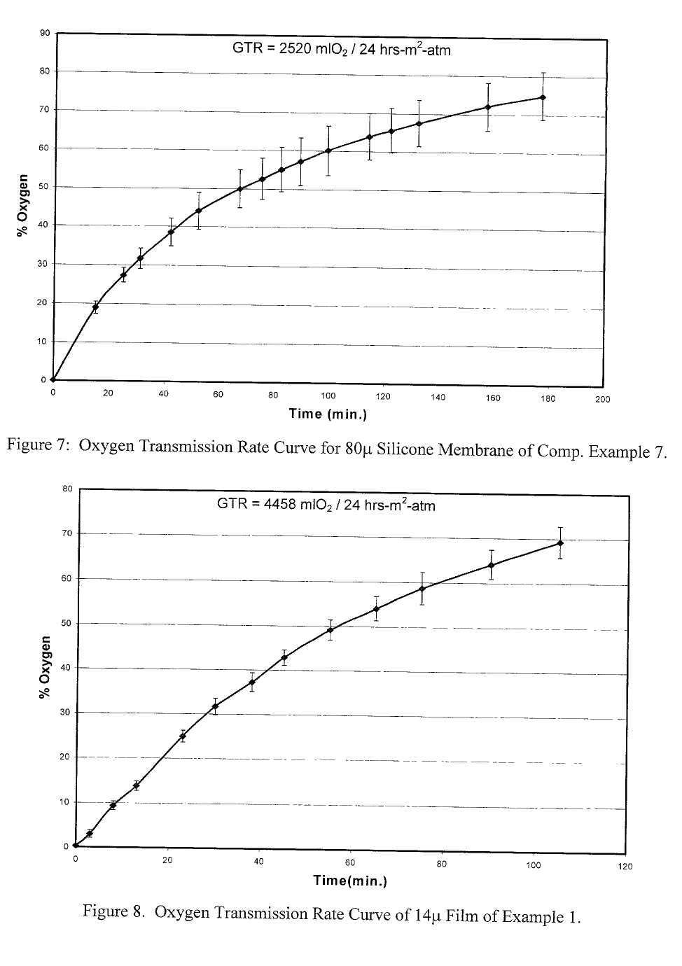

The difference in slopes of the permeability curves for the same type film of differing thicknesses was predictable. Film thickness is one of the primary determinants to gas permeability in any system and is displayed accordingly in the Figures. The GTR values located next to each curve were calculated for measured oxygen content values of the lower chamber above 50% except for the films of Example 4, which never reached 50% before the test was stopped. This becomes very crucial in the determination of a potential membrane for use in a blood oxygenator. Most GTR values for membranes are recorded for the entire range of measure oxygen content, but this is misleading and inaccurate. When used as a membrane for a blood oxygenator, the greatest oxygen difference that will ever be seen across the membrane is near 50%. This is because a person's venous blood oxygen saturation rarely if ever falls below 50%. Since the maximum oxygen percentage that can be used to oxygenate the blood is 100%, this leaves a maximum oxygen differential across the membrane of less than 50%. The importance of this restriction in determining the GTR is seen when the membranes made in accordance with this invention are compared to the membrane of Comparative Example 7, the silicone membrane found in a commercially available oxygenator, Avecor's ECMO 0800, described above. This was selected as the standard because it is the only true membrane used in blood oxygenators to date. The oxygen permeability testing performed on this membrane yielded an excellent GTR at oxygen differentials greater than 60%. However, the GTR was found to decrease significantly as the oxygen differential decreased as is shown in FIGS. 6 and 7.

Although silicone possesses an excellent permeability to oxygen when large differences in partial pressure are present, it declines as the burden to transfer large amounts of oxygen remains steady even as the oxygen difference declines. This is seen clinically by the large surface areas required by this line of oxygenator in order to adequately support patients requiring cardiopulmonary support.

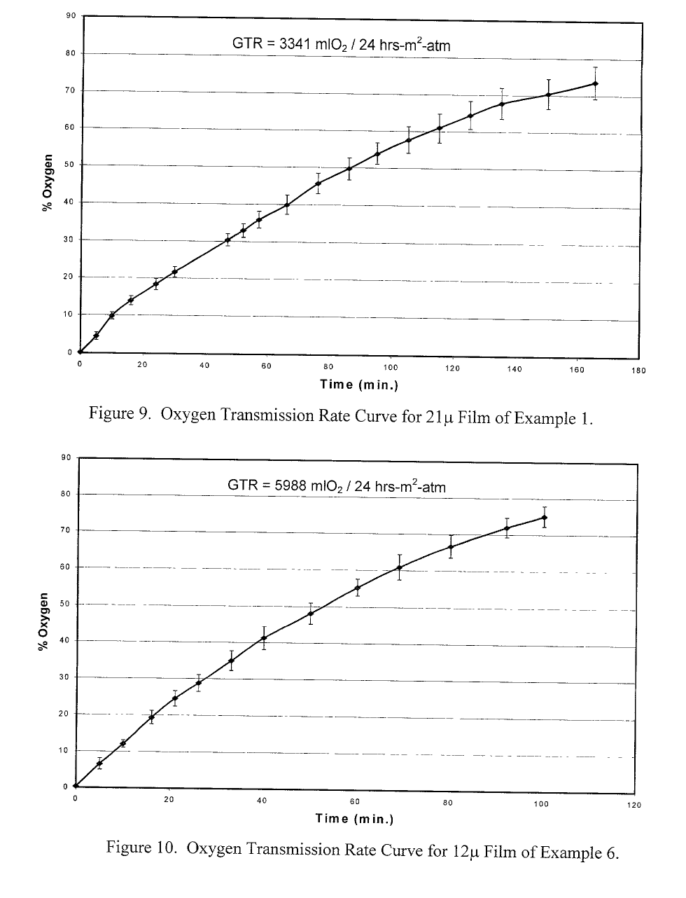

The next set of films to be made and tested were of a softer 3078 Hytrel® (the films of Example 1, with permeability performance illustrated in FIGS. 8 and 9). These films were made due to the low GTR and poor permeability demonstrated by the 4056 Hytrel®. The soft Hytrel® possessed greater oxygen permeability and yielded GTRs that were comparable to those seen with the silicone membranes when the thickness was 14μ.

With both the Hytrel® and Tecoflex® polymers, the diffusion of oxygen was expected to increase with an increase in the polyether content (the soft segment). However, for the examined systems where the hardness of Hytrel® and Tecoflex® were similar, Hytrel® displayed a higher permeability to oxygen than Texcoflex®. One explanation is that the association of the hard segment through hydrogen bonding (as in Tecoflex®) compromises oxygen diffusion in a bi-component system to a greater extent than in the crystallized chains (as in Hytrel®). However, the contribution of differing micro-structures in the polymers, which has not been disclosed by the manufacturers, should not be ignored.

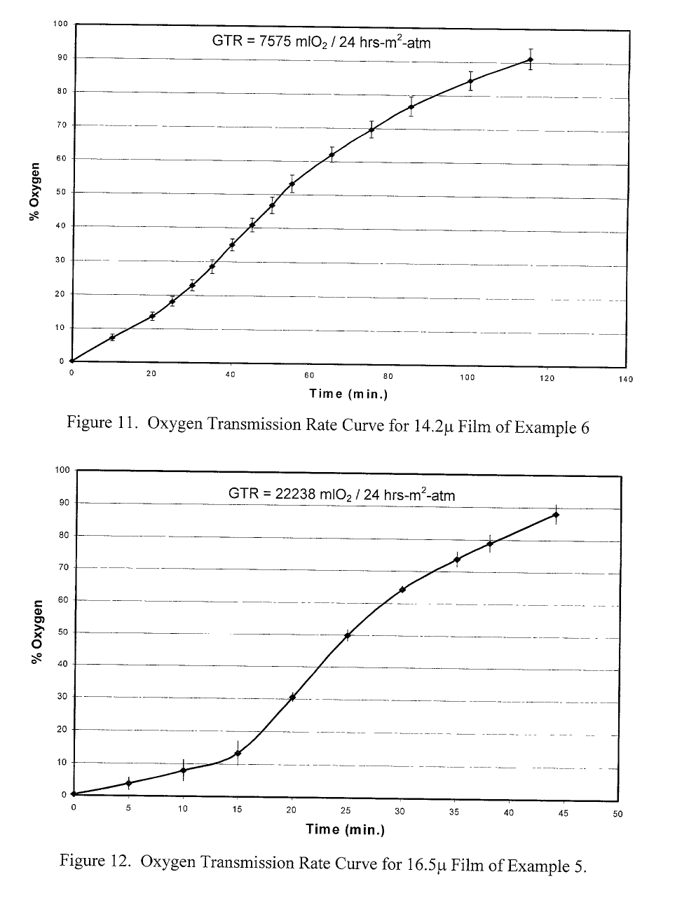

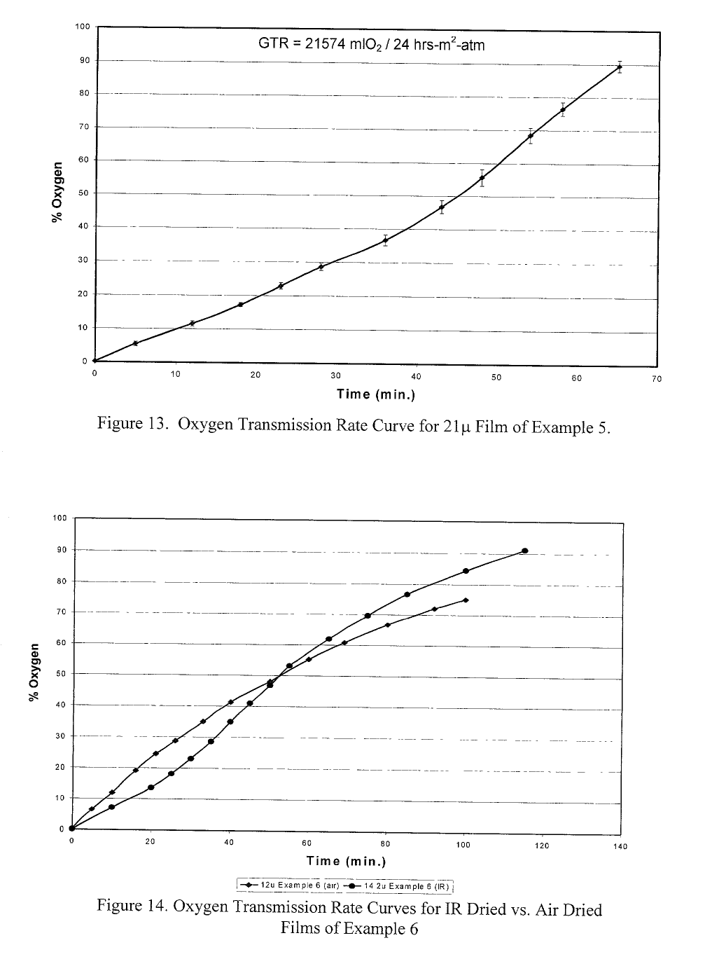

In an attempt to maximize the permeability of Kraton®, the concept of plasticization as a means to improve gas diffusion was explored. Thus plasticized Kraton® 2104 (Example 5) was compared with a neat unplasticized Kraton® 1101 (Example 6). Both Kraton® films possessed excellent permeabilities and GTRs that remained high even with decreasing partial pressures of oxygen across the film as is shown in FIGS. 10-13.

The GTR of the 2104 Kraton® film, the film of Example 5, started off very close to that of the 1101 Kraton® film of Example 6, but started increasing sharply after approximately 15 minutes and did not decrease significantly up to the endpoint of the test at 90% oxygen in the bottom cell. The sharp increase in the GTR is not completely understood but may be related to a saturation phenomenon that occurs in the bulk material of the film. After becoming saturated, the resistance to oxygen diffusion is greatly reduced and allows for an exceptionally high permeability and GTR. The difference between the air-dried and IR dried films was noticed as a 20 minute lag time for the one dried with the IR method as is shown in FIG. 1, discussed above. Although the final GTR was very similar for the two films, the rate of increase in the GTR for the air-dried film was much greater after the initial 15 minutes. The 21μ film did however reach a final GTR that was very close to that of the 16.5μ film as is shown in FIG. 4. This again may be explained by the formation of a skin on the surface of the film when dried using the IR lamp. The GTR of the 1101 Kraton® film of Example 6 was not as high as the 2104 Kraton® film of Example 5 but still surpassed the Avecor silicone membrane (Comparative Example 7) and presents as a viable candidate for the composite membrane construct. The process of using the IR lamp for drying resulted in similar gas transmission for the 1101 Kraton® films as well. Although not as pronounced as with the films of Example 5, there was a definite lag in initial GTR that ended with the 14.2μ film dried with IR having a GTR greater than that of the 12.0μ film as is shown in FIG. 14.

The use of the IR lamp to dry the Kraton® films appeared to have caused an initial resistance to oxygen permeability but resulted in GTRs for thicker films that are equal to or greater than the thinner films that are air dried.

Oxygen Permeability and Oxygen Transmission Rate of Composite Membranes

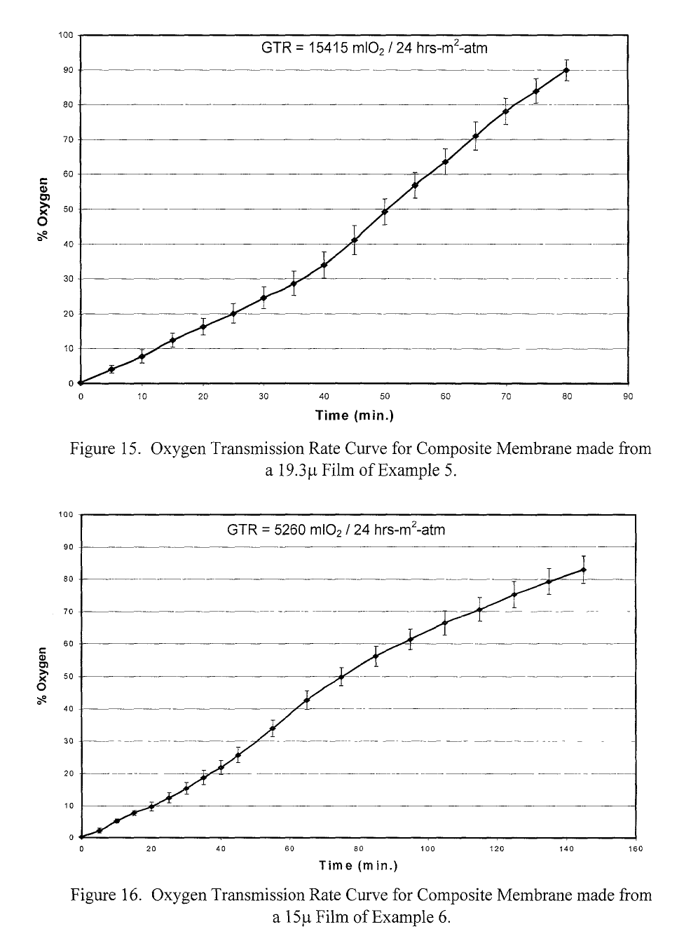

The films of Examples 5 and 6, i.e., those with the highest gas transmission rates were selected to be combined with an open weave polyester fabric for the construction of composite membranes in accordance with the present invention. The resulting permeability curves for the reinforced composites are shown in FIGS. 15 and 16.

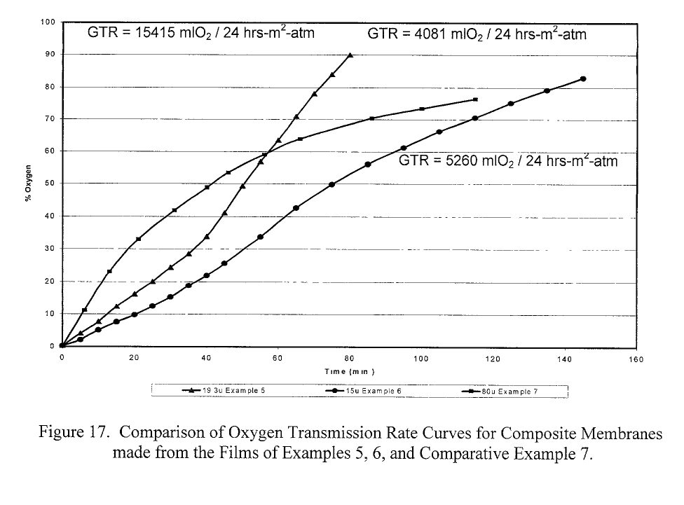

The addition of the fabric support matrix decreased the GTR of both the membranes of Examples 5 and 6 by roughly 30%. The decrease is most likely due to the loss of effective surface area that can participate in the diffusion of oxygen. More specifically, the crystalline nature of the support fabric would expectedly behave as a substantial barrier to diffusion and compromise the overall permeability of the system. The composite membranes were also pressure tested and performed well. The addition of the loosely woven fabric gave the added strength needed for the membranes to be able to withstand the pressures typically seen during CPB. The woven fabric will also provide a spacer between the layers of the membrane material when folded or rolled to produce a complete gas exchange system for blood oxygenators. The GTRs of the two Kraton® composite gas exchange membranes were superior to that of the silicone membrane of Comparative Example 7 as is shown in FIG. 17.

Evaluation of Hemolytic Index

The evaluation of hemolysis was conducted in order to gain an insight into the biocompatibility of the Kraton® composite membranes made from the films of Examples 5 and 6 as compared to the Avecor silicone membrane of Comparative Example 7. Although hemolysis is but one indicator in this assessment, combined with the platelet adhesion study it provided a critical initial evaluation of the suitability of a material to be in a blood contacting device. The hemolysis testing performed in this study followed the ASTM F756-93 standard for non-extracted polymers evaluation using the static method. The results of the hemolysis testing revealed a statistically significant difference between the membrane of Example 5 and the membranes of Example 6 and Comparative Example 7. As is shown in Table II, the hemolysis index of the membrane of Example 5 was approximately half that of both the membranes of Example 6 and Comparative Example 7. All membranes were found to be non-hemolytic as defined by a hemolysis index below 2.0. This test proves the viability of both of the membranes of Examples 5 and 6 for use in the construction of a blood contacting medical devices from a hemolytic standpoint.

TABLE II
Hemoglobin released and hemolysis index for tested membranes

| | Hemoglobin | |
|---|---|---|
| Membrane Type (n = 6) | Released (mg/ml) | Hemolytic Index |
| Example 5 | 0.29 ± 0.22 | 0.012 ± 0.009 |
| Example 6 | 0.79 ± 0.18 | 0.032 ± 0.007 |
| Comparative Example 7 | 0.72 ± 0.22 | 0.029 ± 0.009 |

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims

What is claimed is:

1. A blood oxygenator membrane comprising a composite comprising a polymeric matrix and a reinforcing fiber-based material, the polymeric matrix comprising a thermoplastic elastomer.

2. A composite membrane as set forth in claim 1 wherein the thermoplastic elastomer is selected from the group consisting of polyolefin-polydiene copolymers, hydrogenated polyolefin-polydiene copolymers, polystyrene, polydiene copolymers, polyether-urethane copolymers, polyether-ester copolymers, polydimethyl siloxane-polyether urethane copolymers, polyether-polyolefin copolymers, and blends thereof.

3. A composite membrane as set forth in claim 1 wherein the reinforcing fiber-based material comprises a fabric selected from the group consisting of woven fabric, non-woven fabric and knitted fabric.

4. A composite membrane as set forth in claim 1 wherein the thermoplastic elastomer comprises a block copolymer of a first monomer selected from the group consisting of diene and a second monomer selected from the group consisting of substituted styrene and unsubstituted styrene.

5. A composite membrane as set forth in claim 1 further comprising a surface comprising covalently bonded moieties selected from the group consisting of phosphonyl groups and sulfonyl groups.

6. A composite membrane as set forth in claim 5 further comprising immobilized bioactive agents ionically bonded to the covalently bonded moieties.

7. A composite membrane as set forth in claim 1 wherein the thermoplastic elastomer is crosslinked.

8. A composite membrane as set forth in claim 1 wherein said blood oxygenator membrane is implantable.

9. A composite membrane as set forth in claim 1 wherein the polymeric matrix is microporous, comprising a continuous cell structure.

10. A composite membrane as set forth in claim 1 wherein said oxygenator membrane comprises a component of a chromatographic device.

11. A composite membrane as set forth in claim 1 wherein said oxygenator membrane comprises a component of a dialysis unit.

12. A composite membrane as set forth in claim 1 wherein the thermoplastic elastomer is plasticized.

13. A method for making a blood oxygenator membrane comprising casting a solution of a thermoplastic elastomer onto a fiber-based material.

14. The method set forth in claim 13 wherein the thermoplastic elastomer is selected from the group consisting of polyolefin-polydiene copolymers, hydrogenated polyolefin-polydiene copolymers, polystyrene, polydiene copolymers, polyether-urethane copolymers, polyether-ester copolymers, polydimethyl siloxane-polyether urethane copolymers, polyether-polyolefin copolymers, and blends thereof.

15. The method set forth in claim 13 wherein the fiber-based material comprises a fabric selected from the group consisting of woven fabric, non-woven fabric and knitted fabric.

16. The method set forth in claim 13 further comprising the step of crosslinking the thermoplastic elastomer subsequent to the casting step.

17. A method for making a blood oxygenator membrane comprising the steps of:
a) forming a preliminary polymeric matrix membrane comprising a thermoplastic elastomer;
b) providing a fiber-based material; and
c) applying the polymeric matrix web onto the fiber-based material under heat and pressure.

18. The method set forth in claim 17 wherein the thermoplastic elastomer is selected from the group consisting of polyolefin-polydiene copolymers, hydrogenated polyolefin-polydiene copolymers, polystyrene, polydiene copolymers, polyether-urethane copolymers, polyether-ester copolymers, polydimethyl siloxane-polyether urethane copolymers, polyether-polyolefin copolymers, and blends thereof.

19. The method set forth in claim 17 wherein the fiber-based material comprises a fabric selected from the group consisting of woven fabric, non-woven fabric and knitted fabric.

20. The method set forth in claim 17 further comprising the step of crosslinking the thermoplastic elastomer subsequent to the step of applying the polymeric matrix web to the fiber-based material.
